(19) United States
(12) Patent Application Publication
(10) Pub. No.: US 2017/0014652 A1
(43) Pub. Date: Jan. 19, 2017

(54) Wipe, Method for Producing a Wipe, and Method for Cleaning or Moisturizing a Patient's Skin

(71) Applicant: Medline Industries, Inc., Mundelein, IL (US)

(72) Inventors: Sharbel J. Maalouf, Pleasant Prairie, WI (US); Joseph Ceccoli, Farmingville, NY (US); Alana Cecola, Round Lake, IL (US); Maria Miller, Cary, IL (US); Julie Fischer, Smithtown, NY (US)

(21) Appl. No.: 14/799,227

(22) Filed: Jul. 14, 2015

(51) Int. Cl. A61Q 19/00; B08B 1/00; A61K 8/06; A61K 8/89; A61K 8/02

(52) U.S. Cl. CPC A61Q 19/007; A61K 8/89; A61K 8/0208; A61K 8/06; B08B 1/006

(57) ABSTRACT

Disclosed is a cleansing wipe that comprises the hydrophobic cloth and a cleansing or moisturizing composition, the composition including a cleanser and a silicone emulsion. The cleansing or moisturizing composition is aqueous but has sufficient affinity to the cleansing cloth to allow forcibility in transport and handling of the cloth. When applied to the skin of a patient, the silicone is released from the cloth in an amount effective to inhibit trans-epidermal water loss. The emulsion may comprise, for instance, a C₁₄₋₂₂ alcohol or a C₁₂₋₂₀ alkyl glucoside. The cleansing or moisturizing composition may include components such as cleansers and humectants, and may include other ingredients such as skin nourishers.

### FIELD

The disclosure is in the field of patient care products and in various non-exclusive embodiments is specifically directed to a cleansing or moisturizing wipe. The wipe is intended for use to apply a cleansing or moisturizing lotion to the skin of a patient. The lotion may be applied by a caretaker or by the patient himself.

### BACKGROUND

Numerous types of patient care wipes are known in the art. These range generally from mass-marketed consumer wipes for cleansing, such as baby care products, to products intended for the medical industry, such as wipes infused with an antibiotic or other antimicrobial agent. In the production of such wipes, there are sometimes a number of competing purposes. For various reasons, it is desirable to fashion the wipe from a hydrophobic substrate such as a non-woven hydrophobic material. This can present challenges when the lotion to be applied to the skin of the patient is hydrophilic, because it can be difficult to prepare a lotion that has sufficient affinity for the hydrophobic substrate to permit sufficient stability for transport and storage of the wipe.

Also, in some cases it is desirable to apply a lotion that includes hydrophobic materials. In particular, it is known in the art that silicone-containing lotions can have a beneficial effect when applied to the skin of a patient. Silicone is believed to provide a thin barrier on the skin, thereby inhibiting a phenomenon known as trans-epidermal water loss. The trans-epidermal water loss problem is an issue for skin-compromised patients, in particular, elderly patients and infants. It is believed to be difficult to formulate a wipe using a hydrophobic substrate and a composition that includes silicone, because silicone is itself hydrophobic and has affinity to the substrate, thereby making it difficult to release sufficient silicone to provide the trans-epidermal water loss inhibiting effect.

Otherwise, generally it is desirable when applying a lotion to the skin to include components that cleanse, nourish, or moisturize the skin. Not all of these components are necessarily compatible with one another.

### DESCRIPTION OF THE FIGURE

FIG. 1 is a perspective illustration of a plurality of wipes prepared in accordance with the present disclosure.

### DETAILED DESRCRIPTION

Generally speaking, a cleansing wipe or moisturizing wipe may be prepared from a hydrophobic cloth substrate having an aqueous composition disposed thereon. The aqueous composition may be characterized as an aqueous cleansing composition, or an aqueous moisturizing composition, and in some embodiments, the composition may have both cleansing and moisturizing properties and may include ingredients suitable for each of those purposes.

For instance, a cleansing wipe may comprise a hydrophobic cloth and a cleansing composition disposed on the cloth. The cleansing composition may comprise a cleaner and a silicone emulsion, where the emulsion includes an emulsifier that comprises at least one emulsifying agent. The emulsifier is present in the cleansing composition in an amount effective to impart a cleansing composition affinity to the hydrophobic cloth that is sufficient to provide stability for purposes of transport and storage of the wipe. When the wipe is applied to the skin, however, the wipe releases a lotion that comprises silicone in an amount effective to inhibit trans-epidermal water loss. In most embodiments this is deemed to be a lotion that includes at least 1% by weight of silicone. It should be understood that the composition of the lotion that is released from the cloth may not be identical to the cleansing composition used in preparing the cloth, because some of the components of the lotion may have greater affinity to the hydrophobic substrate than others.

In other embodiments, not mutually exclusive with regard to the above, the invention provides a moisturizing wipe that includes a hydrophobic cloth substrate and an aqueous moisturizing composition disposed on the cloth. The moisturizing composition comprises a humectant and a silicone emulsion. Again, the emulsion includes an emulsifier that includes at least one emulsifying agent, the emulsifier being present in an amount effective to impart a moisturizing composition affinity to the hydrophobic cloth that is sufficient to provide stability. When applied to the skin, however, the cleansing wipe releases a lotion that comprises silicone in an amount effective to inhibit trans-epidermal water loss. Again, because of potential differential affinity of the various components of the moisturizing composition, the composition of the lotion that is released from the cloth may not be identical to the moisturizing composition as applied to the cloth. It is contemplated that, in practice, a cleansing wipe will include both a cleanser and a humectant for purposes of both cleansing the skin and providing a moisturizing effect.

Also encompassed in various alternative embodiments of the invention are a process for preparing a cleansing wipe, a process for preparing a moisturizing wipe, a cleansing method, and a moisturizing method. The process for preparing the wipe generally comprises providing a hydrophobic cloth and providing an aqueous cleansing composition or moisturizing composition as discussed hereinabove, and introducing the cleansing or moisturizing composition to the cloth. The cleansing or moisturizing method generally comprises comprising a cleansing wipe as described above and cleansing or moisturizing the skin of a patient using the wipe.

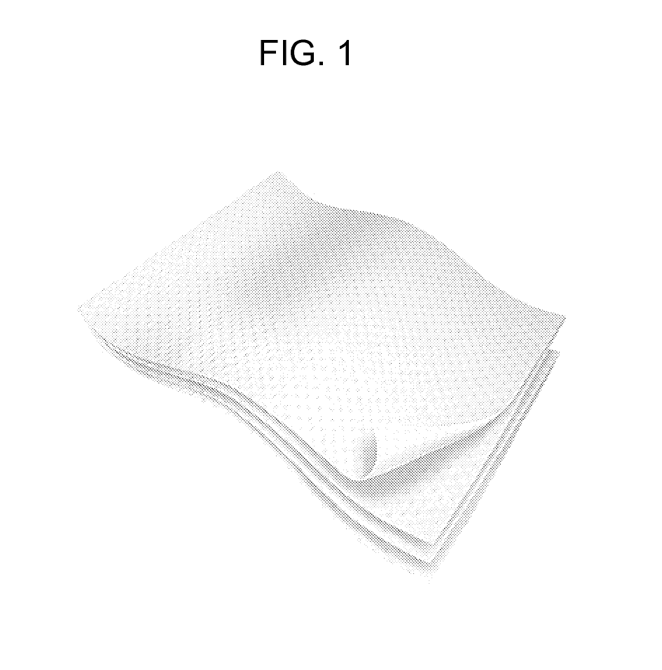

As depicted in FIG. 1, the cloth substrate should be a hydrophobic cloth, by which is contemplated a cloth that is prepared from hydrophobic fibers, either by weaving, or, more preferably, by non-woven methods to provide a non-woven hydrophobic cloth substrate. Any suitable fibers may be employed in the preparation of the substrate, and, for example, the hydrophobic cloth substrate may comprise polyethylene, polypropylene, polyester, or other suitable fibers. The substrate may include other polyolefins, rayons, polyamides, or polyester amides. It is contemplated that a minor portion of the materials of the cloth substrate may be non-hydrophobic. For cost reasons, the substrate preferably is a polyethylene cloth substrate.

The substrate may take any suitable form, but preferably is prepared from a plurality of fibers using a technique suitable for the production of a nonwoven sheet or mat. When fibrous substrate is employed, the fibers may have any suitable fiber length or denier and, for example, the cloth substrate may be a non-woven substrate composed of fibers of 1-4 inches in fiber length, and a denier of 1-5, preferably 2.5-3.8. The wipes may be supplied from the manufacturer in any suitable form. Generally, the wipes will be stacked via conventional mechanical stacking equipment and provided in the form of a package (not shown) that contains plural wipes.

As described hereinabove, disposed on the cloth is a composition that includes cleansing components, moisturizing components, or, preferably, both cleansing and moisturizing components. When the cloth includes a cleansing component, generally, any suitable cleanser can be employed. Preferably, the cleanser is a skin-compatible surfactant, and in some embodiments, and in some embodiments, the surfactant is an amphoteric surfactant. The surfactant may comprise, for example, disodium cocoamphodipropionate. One suitable cleansing agent is MIRANOL® C2M. When employed, the cleansing agent may be present in any suitable amount in the cleansing composition. For instance, the amount of the cleansing composition may be from 0.05%-5% by weight of the composition, more preferably, less than about 1% by weight of the composition.

When the wipe is intended as a moisturizing wipe, the cloth generally includes a humectant that comprises at least one humectifier. The humectant is present in the cleansing composition in an amount of at least 0.5%, which is believed to be an amount suitable to provide skin moisturization, although, more generally, the humectant may be present in any amount suitable to provide skin moisturization. Suitable humectants generally include but are not limited to, polyhydric alcohols, such as glycerin, polyalkalene glycols (such as butylene glycol, propylene glycol, dipropylene glycol, polypropylene glycol, and polyethylene glycol) and derivatives thereof, alkaline polyols and their derivatives, sorbitol, hexylene glycol, 1,3-dibutylene glycol, allantoin, and mixtures thereof. In one embodiment, the humectant comprises three humectifiers, these being glycerin, butylene glycol, and allantoin.

The composition further includes silicone. Any suitable silicone composition may be employed in conjunction with the invention, and the preferred silicone is polydimethyl siloxane (dimethicone). The silicone should be present in an amount effective to provide, in the lotion that is released from the cloth when applying to the skin, an amount of silicone effected to inhibit trans-epidermal water loss. Generally, it is believed that an amount of silicone of about 1% in the lotion will be effective for this purpose. Additionally, the current FDA Monograph for Skin Protectant Drug Products for OTC Human Use in the USA specifies a minimum 1% amount of dimethicone will qualify a product for skin protection. In the composition that is applied to the hydrophobic cloth to form the cloth, the silicone may be present in any suitable amount to attain this release amount, such as an amount ranging from 1-6%, preferably in an amount arranging from about 3-5%. One suitable silicone useful in conjunction with the present invention is XIAMETER® PMX-200 silicone fluid 100CS, available from Dow Corning Corporation.

The silicone is contained the composition in the form of an emulsion and to this end the composition includes an emulsifier, the emulsifier comprising at least one emulsifying agent. The emulsifier is present in the composition in an amount effective to impart affinity of the cleansing or moisturizing composition to the hydrophobic cloth that is sufficient to provide a stable wipe. Generally, the wipe will be deemed stable when at least about 80% of the composition remains on the hydrophobic cloth after 48 hours in static storage.

Any suitable emulsifying agent may be used in conjunction with the invention. Preferable emulsifying agents include fatty alcohols, such as C₁₄₋₂₂ alcohols, or alkyl aldosides, and in particular alkyl glucosides and even more particularly, C₁₂₋₂₀ alkyl glucosides. A particularly preferred emulsifying agent is cetyl alcohol. Other emulsifying agents include MONTANOV L and MONTANOV S, each available from Seppic. MONTANOV L is a proprietary mixture of C₁₄₋₂₂ alcohols and C₁₂₋₂₀ alkyl glucoside for the preparation of a O/W (oil-in-water) emulsion. MONTANOV S is a mixture of coco glucoside and coconut alcohol.

The emulsifier can be present in any amount suitable for the purpose stated above. In some embodiments, the emulsifier can be present in an amount ranging from 1-2% by weight of the cleansing or moisturizing composition. In some cases the emulsifier comprises at least emulsifying agents selected from among the C₁₄₋₂₂ alcohols, C₁₂₋₂₀ alkyl glucosides, coco glucosides, coconut alcohols, and mixtures thereof.

The composition may further include a skin nourisher, by which is contemplated an ingredient that is intended to provide a beneficial effect to the skin other than those described hereinabove with regard to the cleanser, moisturizing agent, and silicone. For example, the skin nourisher may comprise an algae extract, in particular, a Klamath Lake algae extract. The skin nourisher further may comprise, for example, Shea butter. Certain suitable skin nourishers are available from Biocogent LLC under the DERMALRx® product line. These including, for instance, DERMALRx KBGA, a Klamath Lake, blue/green algae extract from the Klamath Lake region in Oregon, DERMALRx® HYDROSEAL CLD, a biofermentation oligopeptide that comprises the fermentation product of a proprietary strain of yeast and DERMALRx® SP, another biofermentation peptide. The Shea butter ingredient may be HEDI® Shea butter E/DU, also from Biocogent LLC. This product comprises a fine micro-dispersion of Shea butter, silicone, urea, and vitamin E. Shea butter is believed to be an emollient and skin conditioner. Notably, HEDI® Shea butter includes silicone and this adds to the silicone content of the composition. When used, the skin nourisher may be present in any suitable amount, for example, an amount ranging from 0.02%-5% by weight, inclusive of all of the skin nourisher ingredients.

The cleansing or moisturizing composition further may include other ingredients such as fragrances, colors, preservatives, and additives to modify the pH. These ingredients may be added in any amounts suitable for their intended purposes. One suitable preservative is EUXYL® PE9010, available from Schülke & Mayr GmbH. This is a liquid cosmetic preservative that is based on phenoxyethanol and ethylhexyl glycerin. The preservative may be present in any amount effective preservative to provide properties. For example, the preservative may be present in an amount of about 0.1%-1.5% by weight.

The composition generally will comprise an oil-in-water (O/W) emulsion having an aqueous phase and a nonaqueous phase. The pH of a cleansing wipe lotion can, if desired, be adjusted to a range compatible with the (average) pH of healthy skin, to a range so as to compensate for the pH of residues that otherwise may remain on the skin after cleansing with a wipe, or to a range more inhospitable to microorganisms. For instance, adjusting the pH to not more than 5.5 or 6 is consistent with the pH of healthy skin being approximately 5. Compensating for residues that may otherwise be alkaline can be achieved by adjusting the pH to a range less than 5, perhaps as low as 4, even down to a pH of 3 in some cases, as may be the case for a lotion less hospitable for certain microorganisms. However, too low a PH can lead to skin irritation. The pH can range from about pH 3 to about pH 9, particularly from about pH 4-pH 7.5.

Compounds for adjusting the pH can be suitably selected from dermatologically acceptable pH control agents, agents suitable for skin care products and the like. Compounds include, for example, fruit acids such as citric acid, conjugate bases like citrates such as sodium citrate or trisodium citrate, gluconic acid, lactic acid, glycolic acid, lactates such as sodium lactates, malic acid, malates such as sodium malate, as well as mixtures of any thereof. The compound(s) to be selected should be compatible with other ingredients in a cleansing wipe lotion.

The composition further may include any suitable thickener. In general, thickeners include certain ingredients that can also serve as thickeners (viscosity-increasing agents). Typically, such viscosity increasing agents include, but are not limited to, hydrogenated vegetable oils like hydrogenated jojoba oil and hydrogenated jojoba wax; microcrystalline wax; paraffin wax; beeswax; carnauba wax; ozokerite wax; ceresine wax; myristyl alcohol; behenyl alcohol; stearyl alcohol; cetearyl alcohol; hydrogels; and mixtures thereof. It will be appreciated that other modifiers that can function as thickeners may be suitably selected.

The selection of a viscosity modifier can influence release of non-hydrophilic ingredients, particularly a silicone (sometimes generally referred to as “dimethicone”), from the wipe substrate. The releasability of the dimethicone is surprisingly improved by incorporating a thickener in the lotion during its preparation. For example, an unexpected increased release of the silicone from a wipe having the lotion is obtainable with a thickener as a modifier which comprises a hydroxyethyl acrylate/sodium acryloyldimethyl taurate copolymer, such as a mixture of hydroxyethyl acrylate/sodium acryloyldimethyl taurate copolymer, isohexadecane, and polysorbate 60, as compared to emulsions currently in the art. An exemplary thickener that surprisingly improves releasability of silicone from a wipe loaded with the lotion includes a mixture commercially available under the brand name SIMULGEL INS 100 from Seppic. This ingredient includes a hydroxyethyl acrylate/sodium acryloyldimethyltaurate copolymer, isohexadecane, and polysorbate 60.

The composition may include other ingredients as suitable for their intended purposes. In preferred embodiments, the composition does not include parabens, nor does it include mineral oil or other petroleum products. These ingredients may be included, however, if desired. Other adjunctive ingredients can include texturizes, anti-oxidants, pH buffers, metal sequestrants, and anti-stick agents. In some embodiments, if desired the composition may include a medically active ingredient, such a biocide.

To prepare the wipe, generally, a cleansing or moisturizing composition as described hereinabove is first prepared, and then is introduced to the hydrophobic cloth substrate. The temperature of mixing during the preparation step can be any suitable temperature, for instance, a temperature ranging from about 80-95 degrees C. The process can involve a cooling step where the temperatures cool to a temperature ranging from about 20-30 degrees C. The hydrophobic cloth substrate may be conventional and generally the process of preparing the wipe may otherwise be conventional except as concerns the cleansing or moisturizing composition that is introduced to the wipe. For example, the hydrophobic cloth substrate may be provided as a roll material which may be cut into wipes of the desired size and packaged in conventional packaging equipment. The matter of introduction of the composition to the cloth substrate may be performed in a bath in any suitable vessel, or otherwise as may be found suitable (for example, by spraying).

In use, a method of treating a patient comprised applying a lotion to the skin of the patient using a wipe as described hereinabove. Because different ingredients in the wipe may be a different level of affinity to the hydrophobic cloth substrate, it is contemplated that the lotion that comes off of the wipe and onto the skin of the patient may be different in composition on an ingredient percentage basis than the cleansing composition that is introduced to the wipe. The lotion may be applied to the skin of the patient by a caretaker or by the patient himself. The patient may be treated by applying the lotion that emanates from the cloth to a desired area of application. This may be done as frequently as desired. For treatment of trans-epidermal water loss it is desirable to treat the patient at least once per day, preferably at least twice per day, and more preferably at least three times per day, for a period of one, two, three or more than three days.

The following properties are deemed particularly desirable for the cleansing or moisturizing composition:
Color: white to off white
Odor: standard; e.g., vanilla
Appearance: standard lotion
pH 4.85-5.85
Specific gravity 0.987-1.020
Viscosity (Brookfield RVT, spindle 2, 50 RPM, one minute, room temperature): 230-250 cps.

The following examples are provided to illustrate the present invention but should not be construed as limiting the invention in the scope.

### EXAMPLES

### Example 1

A composition was prepared from several phases as outlined below:

| Phase | Ingredient | Weight % |
|---|---|---|
| A | Deionized Water | 89.56 |
| B | Glycerin, 99.7% | 1.25 |
| | USP | |
| | Butylene Glycol | 0.5 |
| | Allantoin | 0.08 |
| C | Cetyl Alcohol | 0.1 |
| | MONTANOV L | 1 |
| | MONTANOV S | 0.5 |
| | Soybean Oil, Food | 0.001 |
| | Grade | |
| | LIPOVOL ® SUN | 0.001 |
| | (Sunflower Oil) | |
| | XIAMETER ® PMX- | 3.5 |
| | 200 Silicone Fluid | |
| | 100CS | |
| D | SIMULGEL ™ INS | 0.5 |
| | 100 | |
| E | DERMALRx ® | 0.333 |
| | KBGA | |
| | DERMALRx ® | 0.021 |
| | HYDROSEAL CLD | |
| | DERMALRx ® SP | 0.021 |
| | HEDI ® Shea Butter | 0.5 |
| | E/DU | |
| | EUXYL ® PE 9010 | 1.1 |
| F | MIRANOL ® C2M | 0.833 |
| | Conc. | |
| | Fragrance: | 0.16 |
| | UR233018/00 | |
| | “Vanilla Crème | |
| | INCI 3” | |
| G | Citric Acid | 0.04 |
| | USP/FCC | |
| | Total | 100 |

The composition was prepared in accordance with the following procedure:
Propeller mix phase A to form a vortex.
Add phase B into the propeller mixing phase A.
Phase C ingredients cetyl alcohol, MONTANOV L, and MONTANOV S were then added into the vortex at a temperature of 83-85 degrees C.
Homogenizing mixing was then commenced in the highest sheer mode of the reactor. The mixture was held at temperature without entraining air for 10 minutes.
The remaining phase C ingredients were then added with continued homogenizing.
Forced cool with homogenizing and propeller mixing to 32 degrees C.
Phase D was added and mixing was continued until the mixture was uniform without clumps.
Phase E was then added, followed by phase F.
Forced cool to 25 degrees C.
Changing to propeller mixing only, phase G was sprinkled in until dissolved. The product was the introduced to a polyethylene substrate to form a wipe.

### Example 2

A moisturizing wipe prepared in accordance with Example 1 is used to apply to the skin of a patient.

It is thus seen that the present disclosure provides a moisturizing and cleansing wipe.

Weight percentages of the cleansing composition and lotion as stated herein are on the basis of the entire compositions including water.

Uses of singular terms such as “a,” “an,” are intended to cover both the singular and the plural, unless otherwise indicated herein or clearly contradicted by context. The terms “comprising,” “having,” “including,” and “containing” are to be construed as open-ended terms. Any description of certain embodiments as “preferred” embodiments, and other recitation of embodiments, features, or ranges as being preferred, or suggestion that such are preferred, is not deemed to be limiting. The invention is deemed to encompass embodiments that are presently deemed to be less preferred and that may be described herein as such. All methods described herein can be performed in any suitable order unless otherwise indicated herein or otherwise clearly contradicted by context. The use of any and all examples, or exemplary language (e.g., “such as”) provided herein, is intended to illuminate the invention and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims. Neither the marking of the patent number on any product nor the identification of the patent number in connection with any service should be deemed a representation that all embodiments described herein are incorporated into such product or service.

1. A cleansing wipe comprising:
a hydrophobic cloth; and
an aqueous cleansing composition disposed on said cloth, said cleansing composition comprising a cleanser and a silicone emulsion, said emulsion including an emulsifier that comprises at least one emulsifying agent, said emulsifier being present in an amount effective to impart a cleansing composition affinity to the hydrophobic cloth sufficient to provide a stable cleansing wipe, said cleansing wipe when applied to the skin releasing a lotion that comprises silicone in an amount effective to inhibit trans-epidermal water loss.

2. A cleansing wipe according to claim 1, said cleansing wipe when applied to the skin releasing a lotion that comprises silicone in an amount of at least 1%.

3. A cleansing wipe according to claim 1, said emulsifier comprising at least one emulsifying agent selected from the group consisting of C14-22 alcohols, C12-20 alkyl glucosides, and mixtures thereof.

4. A cleansing wipe according to claim 1, said emulsifier comprising at least one emulsifying agent selected from the group consisting of coco glucosides, coconut alcohols, and mixtures thereof.

5. A cleansing wipe according to claim 1, said emulsifier comprising at least two emulsifying agents selected from the group consisting of C14-22 alcohols, C12-20 alkyl glucosides, coco glucosides, coconut alcohols, and mixtures thereof.

6. A cleansing wipe according to claim 1, said cleanser comprising a surfactant.

7. A cleansing wipe according to claim 6, said surfactant comprising an amphoteric surfactant.

8. A cleansing wipe according to claim 7, said surfactant comprising disodium cocoamphodipropionate.

9. A cleansing wipe according to claim 1, said cleansing composition further including at least one skin nourisher.

10. A cleansing wipe according to claim 9, said skin nourisher comprising an algae extract.

11. A cleansing wipe according to claim 10, said skin nourisher further comprising Shea butter.

12. A cleansing wipe according to claim 1, said cleansing composition further comprising a humectant that comprises at least one humectifier, said humectant being present in said cleansing composition in an amount of at least 0.5%.

13. A cleansing wipe according to claim 12, said humectant comprising at least one humectifier selected from the group consisting of glycerin, butylene glycol, and allantoin.

14. A cleansing wipe according to claim 1, said cleansing wipe when applied to the skin releasing a lotion that comprises silicone in an amount of at least 1%, said emulsifier comprising at least two emulsifying agents selected from the group consisting of C14-22 alcohols, C12-20 alkyl glucosides, coco glucosides, coconut alcohols, and mixtures thereof; said cleanser comprising a an amphoteric surfactant; said cleansing composition further comprising a humectant that comprises at least one humectifier selected from the group consisting of glycerin, butylene glycol, and allantoin, said humectant being present in said cleansing composition in an amount of at least 0.5%.

15. A cleansing wipe according to claim 14, said cleansing composition further including at least one skin nourisher.

16. A cleansing wipe according to claim 15, said skin nourisher comprising an algae extract and further comprising Shea butter.

17. A cleansing wipe according to claim 14, said surfactant comprising disodium cocoamphodipropionate.

18-44. (canceled)

45. A moisturizing wipe comprising:
a hydrophobic cloth; and
an aqueous moisturizing composition disposed on said cloth, said moisturizing composition comprising a humectant and a silicone emulsion, said humectant comprising at least one humectifier, said humectant being present in said cleansing composition in an amount of at least 0.5%, said emulsion including an emulsifier that comprises at least one emulsifying agent, said emulsifier being present in an amount effective to impart a moisturizing composition affinity to the hydrophobic cloth sufficient to provide a stable moisturizing wipe, said cleansing wipe when applied to the skin releasing a lotion that comprises silicone in an amount effective to inhibit trans-epidermal water loss.

46. A moisturizing wipe according to claim 45, said humectant comprising at least one humectifier selected from the group consisting of glycerin, butylene glycol, and allantoin.

47. A moisturizing wipe according to claim 45, said cleansing wipe when applied to the skin releasing a lotion that comprises silicone in an amount of at least 1%.

48. A cleansing wipe according to claim 45, said emulsifier comprising at least one emulsifying agent selected from the group consisting of C14-22 alcohols, C12-20 alkyl glucosides, and mixtures thereof.

49. A moisturizing wipe according to claim 45, said emulsifier comprising at least one emulsifying agent selected from the group consisting of coco glucosides, coconut alcohols, and mixtures thereof.

50. A moisturizing wipe according to claim 45, said emulsifier comprising at least two emulsifying agents selected from the group consisting of C14-22 alcohols, C12-20 alkyl glucosides, coco glucosides, coconut alcohols, and mixtures thereof.

51. A moisturizing wipe according to claim 45, said cleansing composition further including at least one skin nourisher.

52. A moisturizing wipe according to claim 51, said skin nourisher comprising an algae extract.

53. A moisturizing wipe according to claim 52, said skin nourisher further comprising Shea butter.

54-56. (canceled)
